(19) United States
(12) Patent Application Publication
(10) Pub. No.: US 2020/0022743 A1
(43) Pub. Date: Jan. 23, 2020

(54) BONE CEMENT APPLICATOR

(71) Applicant: BIOMET S.A.S., Valence Cedex (FR)

(72) Inventors: Alexis Dupuy, Sancourt (FR); Sebastien Chaligne, Brette Les Pins (FR); Julie Mottet, Chabeuil (FR); Lenaic Giffard, Valence (FR)

(21) Appl. No.: 16/334,861

(22) PCT Filed: Sep. 29, 2017
(86) PCT No.: PCT/EP2017/074898

§ 371 Date: Mar. 20, 2019

Related U.S. Application Data
(60) Provisional application No. 62/401,540, filed on Sep. 29, 2016.
(60) Provisional application No. 62/417,861, filed on Nov. 4, 2016.
(60) Provisional application No. 62/418,431, filed on Nov. 7, 2016.
(60) Provisional application No. 62/426,637, filed on Nov. 28, 2016.

(51) Int. Cl. A61B 17/88

(52) U.S. Cl. CPC A61B 17/8811; A61B 2017/8838; A61B 17/8833; A61B 17/8822

(57) ABSTRACT

Disclosed in an apparatus for applying bone cement. The apparatus can comprise a body and an applicator. The body can include a coupling portion that can be located at a first end of the body and an articulation portion that can be located at a second end of the body. A central axis of the articulation portion can be arranged at an angle relative to a central axis of the body. The applicator can be configured to connect to the articulating portion and pivot about the central axis of the articulation portion.

### RELATED APPLICATION

This application claims priority and is related to U.S. Provisional Patent Application No. 62/417,861, U.S. Provisional Patent Application, 62/418,431, U.S. Provisional Patent Application No. 62/426,637, and U.S. Provisional Patent Application No. 62/401,540, the contents of which are hereby incorporated in their entirety.

### FIELD OF THE DISCLOSURE

The present disclosure relates to surgical implant systems, including implants, instruments, and methods for installing an implant. Specifically, the present disclosure relates to systems and methods for applying bone cement.

### BACKGROUND

The cement components can be combined and mixed under vacuum to minimize bubbles in the paste and reduce the porosity of the resulting cement. If the vacuum is unstable, bubbles can form in different regions of the paste as the components are mixed and polymerize. As the bone cement typically hardens within minutes of combining the components, the paste must typically be formed within the surgical suite immediately before application of the paste to the bone or implant. However, the house vacuum provided in most surgical suites is often too unstable for avoiding forming bubbles in regions of the paste. While separate vacuum equipment within the surgical suite can be provided to create a stable vacuum, the separate vacuum systems are often expensive specialized systems. An inherent drawback is that if any amount of the paste is drawn inadvertently into the house vacuum system or the separate systems, the hardened cement can become hardened within the system requiring extensive repairs or entirely replacing the vacuum system.

A prosthesis or implant can be positioned in an anatomy, such as a human patient, for various purposes. For example, a prosthesis can be positioned to replace an articulating portion of an anatomy. An incorrectly installed or seated implant can result in pain, limit range of motion, increase wear debris, limit stability, and decrease the lifespan of the implant.

### SUMMARY

To better illustrate the system disclosed herein, a non-limiting list of examples is provided here:

Example 1 can include an apparatus for applying bone cement. The apparatus can include a body and an applicator. The body can include a coupling portion located at a first end of the body and an articulation portion located at a second end of the body. A central axis of the articulation portion can be arranged at an angle relative to a central axis of the body. The applicator can be configured to connect to the articulating portion and pivot about the central axis of the articulation portion.

In Example 2, the apparatus of Example 1 can optionally include the applicator being configured to pivot at least 180 degrees about the central axis of the articulation portion.

In Example 3, the apparatus of any one of or any combination of Examples 1 and 2 can optionally include the central axis of the articulation portion being perpendicular to the central axis of the body.

In Example 4, the apparatus of any one of or any combination of Examples 1-3 can optionally include an interior surface of the body defining a constriction proximate the articulation portion.

In Example 5, the applicator of the apparatus of any one of or any combination of Examples 1-4 can optionally include a diffusor.

In Example 6, the apparatus of any one of or any combination of Examples 1-5 can optionally include the applicator including a central axis arranged at an angle relative to the central axis of the articulation portion.

In Example 7, the apparatus of Example 6 can optionally include the angle of the central axis of the articulation portion relative to the central axis of the body being about 90 degrees.

In Example 8, the apparatus of any one of or any combination of Examples 1-7 can optionally include the applicator including an interior surface at least partially contoured to match a contour of a resected bone.

In Example 9, the apparatus of any one of or any combination of Examples 1-8 can optionally include the applicator being selected from a plurality of applicators, each of the plurality of applicators having at least one of a different size or a different shape.

Example 10 can include apparatus for applying bone cement. The apparatus can comprise a body and an applicator. The body including a coupling portion located at a first end of the body and an articulation portion located at a second end of the body. A central axis of the articulation portion can be arranged perpendicular to a central axis of the body. The applicator can be configured to connect to the articulating portion and pivot about the central axis of the articulation portion.

In Example 11, the apparatus of Example 10 can optionally include the applicator being configured to pivot at least 180 degrees about the central axis of the articulation portion.

In Example 12, the apparatus of any one of or any combination of Examples 10 and 11 can optionally include an interior surface of the body defining a constriction proximate the articulation portion.

In Example 13, the applicator of the apparatus of any one of or any combination of Examples 10-12 can optionally include a diffusor.

In Example 14, the apparatus of any one of or any combination of Examples 10-13 can optionally include the applicator including an interior surface at least partially contoured to match a contour of a resected bone.

In Example 15, the apparatus of any one of or any combination of Examples 10-14 can optionally include the applicator being selected from a plurality of applicators, each of the plurality of applicators having at least one of a different size or a different shape.

Example 16 can include a method for applying bone cement. The method can comprise: pivoting an applicator into a desired angle relative to central axis of an articulation portion, the articulation portion connected to a body at a 90 degrees angle, the articulation portion connected to the applicator; contacting a surface of a first bone with the applicator; and forcing the bone cement into pores of the first bone via a pressure generated within the body due to the applicator being in contact with the surface of the first bone.

In Example 17 , the method of Example 16 can optionally include pivoting the applicator to the desired angle including pivoting the applicator to an angle between +/−90 degrees relative to a central axis of the body.

In Example 18, the method of any one of or any combination of Examples 16 and 17 can optionally include selecting the applicator from a plurality of applicators.

In Example 19, the method of any one of or any combination of Examples 16-18 can optionally include placing the applicator in between the first bone and a second bone, the first bone and the second bone adjacent to one another.

In Example 20, the method of Example 19 can optionally include a first bone is a femur and a second bone is a tibia.

Example 21 can include an apparatus for applying bone cement. The apparatus can comprise a body and an applicator. The body can include a coupling portion located at a first end, an articulation portion located at a second end, and an interior surface defining a through passage connecting the coupling portion and the articulation portion. The applicator can be connected to the articulating portion and configured to articulate with at least two degrees of freedom.

In Example 22, the apparatus of Example 21 can optionally include the articulating portion including a rounded profile.

In Example 23, the apparatus of any one of or any combination of Examples 21 and 22 can optionally include an indentation extending into an outer surface of the body and located proximate the articulation portion, the indentation configured to limit motion of the applicator with respect to one of the at least two degrees of freedom.

In Example 24, the apparatus of any one of or any combination of Examples 21-23 can optionally include one of the at least two degrees of freedom including rotation about a central axis of the body.

In Example 25, the apparatus of any one of or any combination of Examples 21-23 can optionally include one of the at least two degrees of freedom including rotation about a center point of the articulation portion.

In Example 26, the apparatus of Example 25 can optionally include the rotation being limited to +/−20 degrees relative to a central axis of the body.

In Example 27, the apparatus of any one of or any combination of Examples 21-26 can optionally include the interior surface defining a constriction proximate the articulation portion.

In Example 28, the apparatus of any one of or any combination of Examples 21-27 can optionally include the applicator being a diffusor.

In Example 29, the apparatus of any one of or any combination of Examples 21-27 can optionally include the applicator being a nozzle.

In Example 30, the apparatus of Example 29 can optionally include the applicator including a length substantially longer than a diameter of an interior passage of the nozzle.

In Example 31, the apparatus of Example 29 can optionally include the applicator including an exit that has a diameter that is larger than the constriction defined by the body.

Example 32 can include an apparatus for applying bone cement. The apparatus can comprise a body and an applicator. The body can include an inner surface and an exterior surface. The inner surface defining a circular through passage having an inlet and an exit. The exit having an exit diameter and the inlet having an inlet diameter. The exit diameter being smaller than the inlet diameter. The exterior surface defining an articulation portion proximate the exit. The articulation portion having a circular profile. The applicator connected to the articulating portion and configured to articulate relative to a longitudinal axis of the circular through passage.

In Example 33, the apparatus of Example 32 can optionally include the exterior surface defining an indentation proximate the articulation portion, the indentation configured to limit motion of the applicator.

In Example 34, the apparatus of any one of or any combination of Examples 32 and 33 can optionally include the applicator being rotatable about the longitudinal axis of the circular through passage.

In Example 35, the apparatus of any one of or any combination of Examples 32-34 can optionally include the articulation of the applicator relative to the longitudinal axis of the circular through passage being limited to +/−20 degrees.

In Example 36, the apparatus of any one of or any combination of Examples 32-35 can optionally include the interior surface defining a constriction proximate the exit.

In Example 37, the apparatus of any one of or any combination of Examples 32-36 can optionally include the applicator being a diffusor.

In Example 38, the apparatus of any one of or any combination of Examples 32-37 can optionally include the applicator being a nozzle.

In Example 39, the apparatus of Example 38 can optionally include the nozzle including a length substantially longer than a diameter of the nozzle.

Example 40 can include a method for applying bone cement. The method can comprise: pivoting an applicator into a desired angle relative to a central axis of a body connected to the applicator; contacting a surface of a resected bone with the applicator; and forcing the bone cement into pores of the resected bone via a pressure generated within body due to the applicator being in contact with the surface of the resected bone.

In Example 41, the method of Example 41 can optionally include pivoting the applicator to the desired angle including pivoting0the applicator between about 0 degrees and about 20 degrees relative to the central axis.

Example 42 is a system for producing reduced porosity bone cement, comprising: a vacuum pump module comprising: a housing defining an internal chamber and at least one outlet opening, an inlet connector defining an inlet opening through the housing into the internal chamber, and a vacuum connector defining a vacuum outlet through the housing into the internal chamber, wherein the vacuum pump module is configured to receive an airflow through the inlet opening into the internal chamber and exiting through the at least one outlet opening to draw air through the vacuum opening; and a mixing device including a mixing container defining a mixing space; wherein the mixing container is fluidly connected to the vacuum connector such that drawing air through the vacuum opening creates a vacuum within the mixing space at a predetermined pressure.

In Example 43, the subject matter of Example 42 optionally includes wherein the vacuum pump module can further comprise: a venturi element defining a channel extending through the internal chamber from the vacuum outlet to a vacuum opening; wherein the vacuum opening is proximate to the inlet opening such that the airflow entering through the inlet opening and passing the vacuum opening draws air through the channel and from the vacuum outlet.

In Example 44, the subject matter of any one or more of Examples 42-43 optionally include wherein the airflow enters the inlet opening at an input pressure of about 3 bar to about 9 bar.

In Example 45, the subject matter of any one or more of Examples 42-44 optionally include wherein the predetermined pressure of the created vacuum is between about −0.60 bar to about −0.90 bar.

In Example 46, the subject matter of any one or more of Examples 42-45 optionally include wherein the vacuum pump module comprises an injection molded polymer.

In Example 47, the subject matter of any one or more of Examples 42-46 optionally include wherein the mixing space has wall surfaces to which clumps of bone cement adhere during mixing of components of the bone cement.

In Example 48, the subject matter of Example 47 optionally includes wherein the mixing device further comprises: means for mixing the components in the mixing space; a front member provided at a front end of the mixing cylinder, the front member having a discharge opening for discharging bone cement from the mixing space; and a movable piston fixedly mounted at a rear end of the mixing cylinder, the movable piston being releasable from the rear end of the mixing cylinder and movable in the mixing space toward the discharge opening for collecting the clumps of bone cement adjacent the discharge opening, and for discharging bone cement through the discharge opening; wherein, when the movable piston is released from the rear end of the mixing cylinder and moved in the mixing space for collecting the clumps of bone cement adjacent to the discharge opening such that the vacuum generated in the mixing space by the vacuum pump module generates a vacuum in pores formed in the bone cement during the collecting of the clumps of bone cement, whereby the size of the pores is reduced when the bone cement, after collecting, is exposed to atmospheric pressure.

In Example 49, the subject matter of Example 48 optionally includes wherein the vacuum connector of the vacuum pump module is connected to the front member.

In Example 50, the subject matter of Example 49 optionally includes wherein the front member is a cap adapted to seal the mixing space at the front end of the mixing cylinder, and an aperture for the mixing means is provided in the cap; the discharge opening has a connecting-pipe section; and a hose extending from the vacuum connector of vacuum pump module is connected to the connecting-pipe section.

In Example 51, the subject matter of Example 50 optionally includes wherein the connecting-pipe section forms a part of a plug which is located in a tube section provided on the cap.

In Example 52, the subject matter of Example 51 optionally includes wherein the tube section is provided with external threads, and the plug has a flange with internal threads for engagement with the external threads.

In Example 53, the subject matter of any one or more of Examples 48-52 optionally include wherein the movable piston is moved in the mixing space toward the discharge opening by the vacuum generated by the vacuum pump module.

In Example 54, the subject matter of any one or more of Examples 48-53 optionally include wherein the movable piston comprises at least one gripping portion and the mixing cylinder comprises at least one corresponding gripping portion, the gripping portions of the movable piston and the mixing cylinder are engaged with each other for fixing the movable piston to the mixing cylinder, and the gripping portions are disengaged from each other by at least one of: rotating the movable piston relative to the mixing cylinder and rotating the mixing cylinder relative to the movable piston.

In Example 55, the subject matter of Example 54 optionally includes wherein the gripping portions are disengaged from each other by rotating the mixing cylinder relative to the movable piston after the movable piston is placed into engagement with a separate member, the separate member prevents the movable piston from rotating when the mixing cylinder is rotated relative to the separate member.

In Example 56, the subject matter of any one or more of Examples 48-55 optionally include wherein a discharge pipe for discharging bone cement from the mixing space is provided on a tube section formed on the front member.

In Example 57, the subject matter of Example 56 optionally includes wherein the discharge pipe is provided with internal threads for engagement with external threads provided on the tube section.

Example 58 is a method for producing reduced porosity bone cement by mixing components forming a part thereof, comprising: connecting an inlet opening through a housing of a vacuum pump module to an air supply, wherein the housing defines at least one outlet opening and a vacuum outlet; supplying an airflow from the air supply into the inlet opening and exiting through the at least one outlet opening to draw air through the vacuum opening; connecting a mixing device to the vacuum outlet of the vacuum pump module, the mixing device including a mixing container defining a mixing space, wherein drawing air through the vacuum opening creates a vacuum within the mixing space at a predetermined pressure; and mixing the components under vacuum in the mixing space of the mixing device.

In Example 59, the subject matter of Example 58 optionally includes wherein the vacuum pump module can further comprise: a venturi element defining a channel extending through the internal chamber from the vacuum outlet to a vacuum opening.

In Example 60, the subject matter of Example 59 optionally includes wherein the airflow is supplied to the inlet opening at an input pressure of about 3 bar to about 9 bar.

In Example 61, the subject matter of any one or more of Examples 58-60 optionally include wherein the predetermined pressure of the created vacuum is between about −0.60 bar to about −0.90 bar.

In Example 62, the subject matter of any one or more of Examples 58-61 optionally include wherein the vacuum pump module comprises an injection molded polymer.

In Example 63, the subject matter of any one or more of Examples 58-62 optionally include wherein clumps of bone cement adhere to an inner wall surface of the mixing space.

In Example 64, the subject matter of Example 63 optionally includes collecting the clumps of bone cement in the mixing space close to a discharge opening which is adapted to discharge bone cement from the mixing space, wherein the collecting step includes the steps of: exposing the bone cement in the mixing space to vacuum, and moving a piston through the mixing space towards the discharge opening to collect the bone cement near the discharge opening, thereby providing vacuum in those pores in the bone cement which is formed during collection of the bone cement clumps, whereby the pores formed in the bone cement are reduced in size when the bone cement is ultimately subjected to atmospheric pressure after collection.

In Example 65, the subject matter of Example 64 optionally includes wherein the collecting step is accomplished by maintaining the vacuum after the mixing of bone cement has been completed.

In Example 66, the subject matter of any one or more of Examples 64 and 65 optionally include wherein the collecting step is accomplished under at least 40% vacuum.

In Example 67, the subject matter of any one or more of Examples 64-66 optionally include wherein the mixing step is accomplished in the mixing space of the mixing device which is adapted to apply the bone cement, and the collecting step is accomplished by moving the piston by generating a vacuum in the mixing space, the method further comprising the step of applying the bone cement by pressing the bone cement out of the mixing space through further movement of the piston.

In Example 68, the subject matter of Example 67 optionally includes wherein the mixing step is accomplished by fixing the piston at one end of the mixing space opposite the discharge opening, and wherein the collecting step is accomplished by releasing the piston such that the piston can be moved toward the discharge opening for collection of the bone cement.

In Example 69, the subject matter of Example 68 optionally includes releasing the piston in the collecting step by rotating the piston relative to the mixing device.

In Example 70, the subject matter of Example 69 optionally includes connecting step is accomplished by connecting the vacuum pump module to the mixing device through the discharge opening during the mixing and collecting steps, and pressing the bone cement out of the discharge opening during the applying step.

In Example 71, the subject matter of Example 70 optionally includes wherein the applying step is accomplished by connecting a discharge pipe to the mixing device through the discharge opening for discharging bone cement through the discharge opening and discharge pipe, and providing the mixing device with means for mixing the bone cement, the mixing means including an operating rod which protrudes from the mixing device in a same direction as the discharge pipe, which operating rod is broken adjacent to the mixing device prior to the applying step.

In Example 72, the subject matter of any one or more of Examples 64-71 optionally include wherein the collecting step is accomplished under about 80% to about 95% vacuum.

In Example 73, the apparatuses or methods of any one of or any combination of Examples 1-72 is optionally configured such that all elements or options recited are available to use or select from.

### BRIEF DESCRIPTION OF THE FIGURES

The above-mentioned and other features and advantages of this disclosure, and the manner of attaining them, will become more apparent and the disclosure itself will be better understood by reference to the following description of embodiments taken in conjunction with the accompanying drawings, wherein:

FIGS. 1A and 1B show an example of an apparatus for applying bone cement in accordance with at least one example of the present disclosure.

FIG. 2 shows an example of an apparatus for applying bone cement in accordance with at least one example of the present disclosure.

FIG. 3 shows an applicator in accordance with at least one example of the present disclosure.

FIG. 4 shows an example method for applying bone cement in accordance with at least one example of the present disclosure.

FIGS. 5A-5E show an exemplary process for applying bone cement in accordance with the method shown in FIG. 4.

FIGS. 6A and 6B show an example of an apparatus for applying bone cement in accordance with at least one example of the present disclosure.

FIGS. 7A and 7B show an example of an apparatus for applying bone cement in accordance with at least one example of the present disclosure.

FIG. 8 shows an example method for applying bone cement in accordance with at least one example of the present disclosure.

FIG. 9 shows an example of an apparatus for applying bone cement in accordance with at least one example of the present disclosure.

FIG. 10 shows an example of an apparatus for applying bone cement in accordance with at least one example of the present disclosure.

FIG. 11 shows an example assembly process for an apparatus for applying bone cement in accordance with at least one example of the present disclosure.

FIG. 12 shows an example method for applying bone cement in accordance with at least one example of the present disclosure.

FIGS. 13A and 13B show an exemplary process for applying bone cement in accordance with the method shown in FIG. 4.

FIG. 14 show a schematic view of a system for combining and mixing components of a bone cement under vacuum according to an example of the present disclosure.

FIG. 15 show a perspective view of a vacuum pump module according to an example of the present disclosure.

FIGS. 16A and 16B show a cross-sectional side view of a vacuum pump module according to an example of the present disclosure.

FIG. 17 is a perspective view of a vacuum pump module with an attached input line according to an example of the present disclosure.

FIG. 18 is a cross-sectional side view of a mixing device according to an example of the present disclosure.

FIG. 19 is a schematic view of a front portion of the mixing device according to an example of the present disclosure.

FIG. 20 is a schematic view of the mixing system of FIG. 14 filled with components for producing bone cement in vacuum according to an example of the present disclosure.

FIG. 21 is a schematic view of the mixing system of FIG. 14 after mixing the components such that bone cement is produced according to an example of the present disclosure.

FIG. 22 is a schematic view of the mixing system of FIG. 14 during collection of the bone cement produced according to an example of the present disclosure.

FIG. 23 is a schematic view of the mixing system of FIG. 14 after collecting the bone cement and application of a discharge pipe for discharging or ejecting the bone cement according to an example of the present disclosure.

FIG. 24 is a partial schematic view of a rear portion of the mixing device depicted in FIG. 18 according to an example of the present disclosure.

Corresponding reference characters indicate corresponding parts throughout the several views. The exemplifications set out herein illustrate exemplary embodiments of the disclosure, and such exemplifications are not to be construed as limiting the scope of the disclosure any manner.

### DETAILED DESCRIPTION

As used herein, the following directional definitions apply. Anterior and posterior mean nearer the front or nearer the rear of the body, respectively, proximal and distal mean nearer to or further from the root of a structure, respectively, and medial and lateral mean nearer the sagittal plane or further from the sagittal plane, respectively. The sagittal plane is an imaginary vertical plane through the middle of the body that divides the body into right and left halves. In addition, the terms implant and prosthesis, and variations thereof, can be used interchangeably.

During a surgical procedure bone cement can be used to help anchor an implant or other components of a prosthesis onto or within a bone. For example, during a knee surgery, bone cement can be used to help secure a tibial component to a tibia or a femoral component to a femur. To help strengthen the bond between the implant and the bone, the bone cement can be forced into pores of the bone such that upon curing, the bone cement can form a matrix within the bone.

The bone cement can be forced into pores of the bone by placing an applicator in contact with a surface of the bone. The surface can be resected. In order to place the applicator in contact with the surface of the bone, the applicator can be pivoted relative to a body of an apparatus used to apply the bone cement. By pivoting the applicator relative to the body, the applicator can be placed in between bones or other anatomical structures otherwise inaccessible by the applicator.

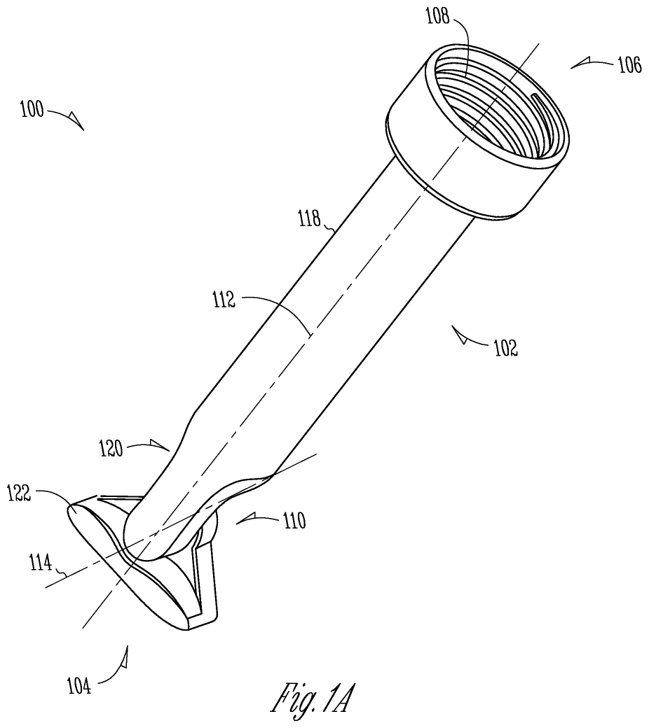

Turning now to the figures, FIGS. 1A and 1B show an example of an apparatus 100 for applying bone cement in accordance with at least one example disclosed herein. The apparatus 100 can include a body 102 and an applicator 104. As shown in FIGS. 1A and 1B, the body 102 can include a coupling portion 106 located at a first end of the body 102. The coupling portion 106 can allow the apparatus 100 to connect to a dispenser that can contain the bone cement. For example, as shown in FIG. 1A, the coupling portion 106 can include female threads 108 (i.e., an internally threaded surface). Other non-limiting examples of mechanisms suitable for use as the coupling portion 106 include Luer lock, male threads, press fit connections, a grove within the body 102 that can receive a peg or other protrusion extending from the dispenser, etc.

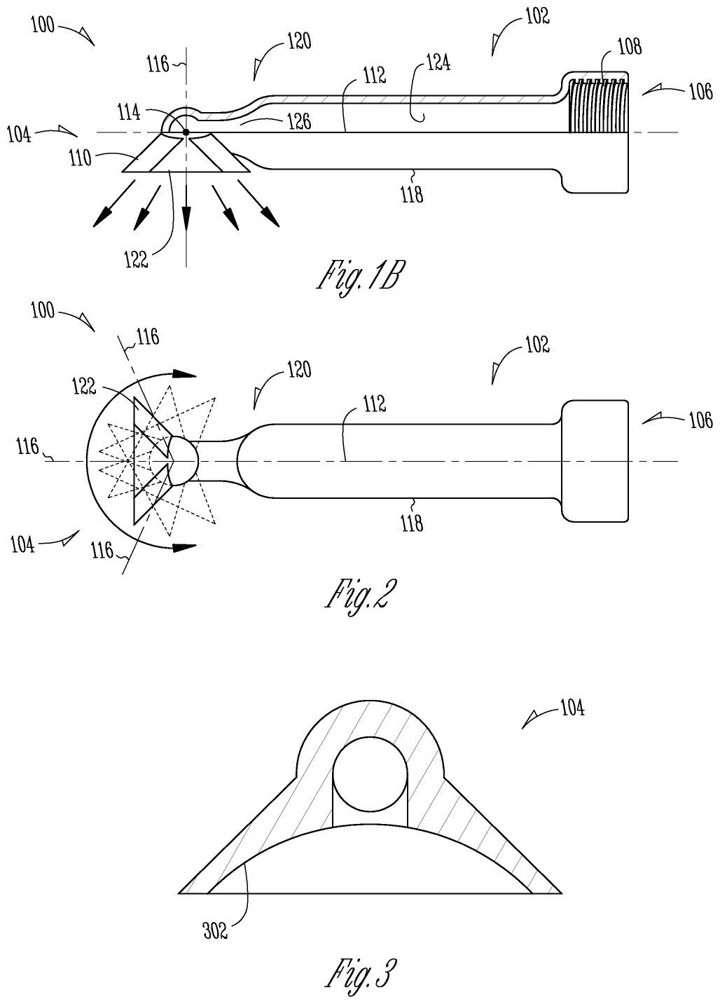

The body 102 can also include an articulation portion 110. The articulation portion 110 can be connected to the coupling portion 106 via a through passage defined by an interior surface. As shown in FIG. 2, the articulation portion 110 can allow the applicator 104 to pivot. In addition, as shown in FIG. 1A, the articulation portion 110 can cause a flow of the bone cement to change directions. For example, the bone cement can initially flow along a longitudinal axis 112 of the body 102. Upon entering the articulation portion 110, the flow of the bone cement can be redirected along an axis 114 and into the applicator 104. Upon entering the applicator 104, the flow of the bone cement can be further redirected along an axis 116.

The position of the axis 116 can be altered by rotation of the applicator 104. For example, as shown in FIG. 2, the axis 116 can be aligned with the longitudinal axis 112 of the body 102. In addition, as the applicator 104 is pivoted about the axis 114, the axis 116 can pivot relative to the longitudinal axis 112. As shown in FIGS. 1A and 1B, the axis 114 can be perpendicular to the longitudinal axis 112.

The articulation portion 110 can be separated from a main section 118 of the body 102. The separation can be delineated by an indentation 120 proximate the articulation portion 110. As described herein, the indentation 120 can define a range of motion of the applicator 104 relative to the body 102. The range of motion of the applicator 104 can vary depending upon an application. For example, various surgical procedures may need a large range of motion and the applicator 104 can pivot between about +/−90° to about +/−135° relative to the longitudinal axis 112. Other applications may not need a large range of motion and the applicator 104 may pivot between about +/−45° relative to the longitudinal axis 112.

The applicator 104, as shown in FIGS. 1A, 1B, and 2, can be in the form of a diffusor. As shown, the applicator 104 can include an exit 122 that is larger than a diameter of the indentation 120. As a result, during a surgical procedure, a surgeon can utilize the applicator 104 to apply bone cement to a portion of bone. Use of the apparatus 100 can allow the surgeon to pressurize the application of the bone cement such that the bone cement is forced into pores of the bone. The pressurization can be caused by a pressure gradient within the bone cement.

As shown in FIG. 1B, the body 102 can include an interior surface 124. The interior surface 124 can define a constriction 126. The constriction 126 can be a curved portion located proximate the articulation portion 110 formed by the interior surface 124. Still consistent with embodiments disclosed herein, the constriction 126 can include the interior surface 120 forming a conical or other shape that has a larger opening proximate the coupling portion 106 than an exit proximate the articulation portion 110.

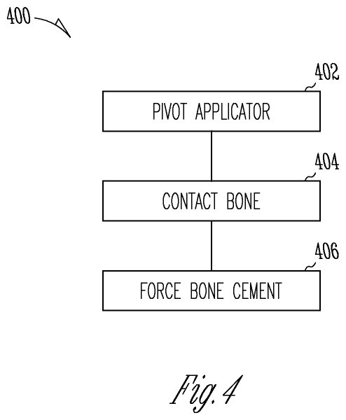

As shown in FIG. 3, the applicator 104 can include an interior surface 302. The interior surface 302 can include a contour that can help the applicator 104 conform to a bone. For example, the interior surface 302 can be rounded to better interface with a portion of a femur proximate a tibia.

The body 102 and the applicator 104 can be manufactured from metals, polymers, ceramics, or any combination thereof. For example, the body 102 can be manufactured from a metal or ceramic and the applicator 104 can be manufactured from a polymer. Manufacturing the applicator 104 from a polymer or other flexible material can allow the applicator 104 to be removed from the body 102. As a result, the apparatus 100 can be a component of a kit or system that can include a plurality of applicators. As a result, a surgeon can select an appropriate applicator from a plurality of applicators for a given surgical procedure or that is sized for a particular patient.

The body 102 and the applicator 104 can be manufactured from various techniques including, but not limited to, machining, injection molding, casting, etc. For example, the body 102 and the applicator 104 can be injection molded. In addition, the body 102 can be machined from a cast billet using a computer numerical controlled (CNC) machine and the applicator 104 can be overmolded onto the articulation portion 108.

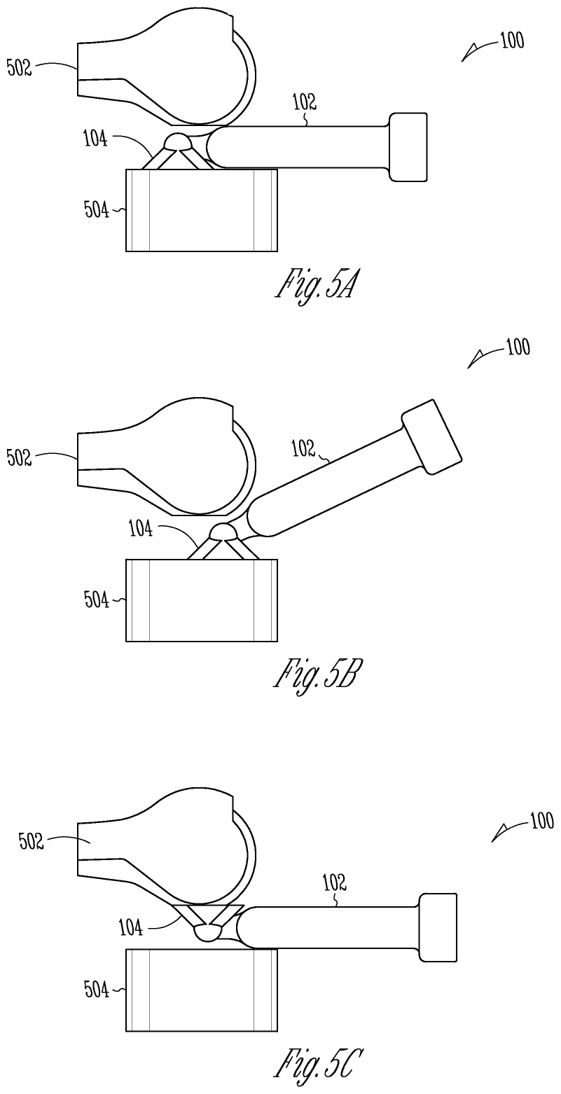

FIG. 4 shows a flowchart for a method 400 for applying bone cement in accordance with at least one example disclosed herein. The method 400 begins at stage 402 where an applicator, such as the applicator 104, of an apparatus, such as the apparatus 100, can be pivoted into a desired angle. For example, the applicator can be pivoted to a desired angle relative to a central axis, such as the longitudinal axis 112, of a body, such as the body 102. For instance, the applicator can be pivoted between +/−90° relative to the central axis.

From stage 402, the method 400 can proceed to stage 404 where the applicator can be placed in contact with a bone. For example, during a knee surgery, the applicator can be positioned such that an exit of the applicator is in contact with a resected surface of a bone. By pivoting the applicator relative to the body, the applicator can be placed in between bones to better contact the resected surface of the bone.

From stage 404, the method 400 can proceed to stage 406 where the bone cement can be forced into pores of the resected bone. The bone cement can be forced into the pores of the bone by creating a pressure gradient within the bone cement. The pressure gradient can force the bone cement into the pores because the flow of the bone cement is restricted to a direction into the pores by the body and applicator of the apparatus.

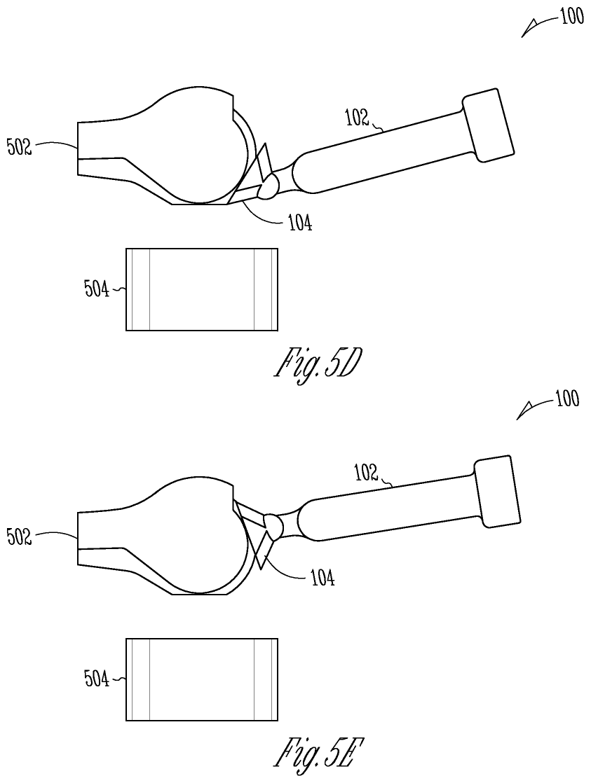

FIGS. 5A-5E show an exemplary process for applying bone cement in accordance with the method 400. As shown in FIGS. 5A-5C, the applicator 104 can be pivoted relative to the body 102 and placed in between a first bone 502 and a second bone 504. The first bone can be a femur and the second bone can be a tibia. By pivoting the applicator 104 relative to the body 102, the apparatus 100 can achieve a form factor that can allow the applicator 104 to be placed in between the first bone 502 and the second bone 504. Once the applicator is in position, bone cement can be applied to the second bone 502 as shown in FIGS. 5A and 5B and the first bone 502 as shown in FIGS. 5C-5E.

Once the applicator 104 is in contact with bone, either the first bone 502 or the second bone 504, the applicator 104 can be slid or otherwise moved across the surface, resected or not, of the bone. As the applicator 104 is being translated across the bone surface, the bone cement can be forced from the applicator 104 into pores of the bone. Also, as shown in FIGS. 5D and 5E, the apparatus 100 can be used to apply bone cement to bone surfaces that do not abut another bone. Rotation of the applicator 104 relative to the body 102 can allow a surgeon to position the applicator 104 for many applications.

As disclosed herein, the applicator 104 being repositionable can allow the surgeon to inject and pressurize cement in a tibial surface after the pressurization on the femoral surface, or vice versa. The size of the nozzle can allow it to fit in a gap between the tibia and femur after resection of the two bones. This can be beneficial during a knee arthroplasty where cross ligament preservation is desired or to otherwise minimize tissue disturbance during surgery.

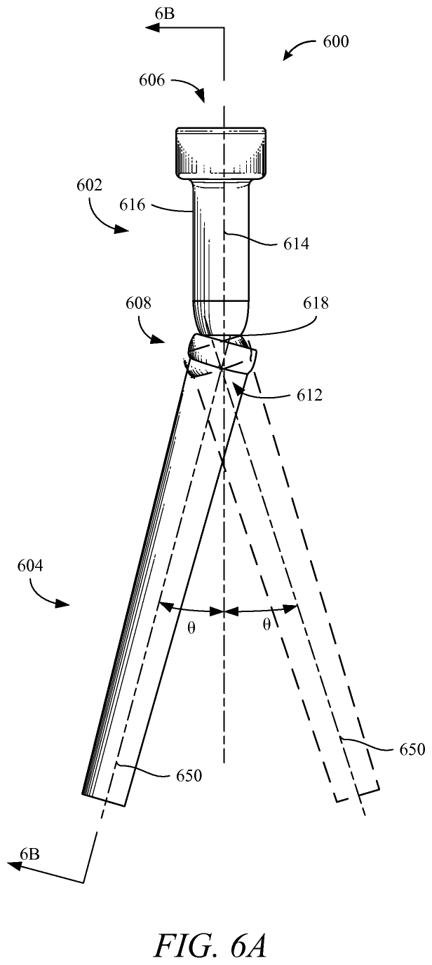

FIGS. 6A and 6B show an example of an apparatus 600 for applying bone cement in accordance with at least one example disclosed herein. The apparatus 600 can include a body 602 and an applicator 604. As shown in FIGS. 6A and 6B, the body 602 can include a coupling portion 606 located at a first end of the body 602. The coupling portion 606 can allow the apparatus 600 to connect to a dispenser that can contain the bone cement. As shown in FIG. 6B, the coupling portion 606 can include female threads 650 (i.e., an internally threaded surface). Other non-limiting examples of mechanisms suitable for use as the coupling portion 606 include a Luer lock, male threads, press fit connections, a grove within the body 602 that can receive a peg or other protrusion extending from the dispenser, etc.

The body 602 can also include an articulation portion 608. The articulation portion 608 can be connected to the coupling portion 606 via a through passage defined by an interior surface 610. The articulation portion 608 can include a rounded profile that can allow the applicator 604 to articulate with at least two degrees of freedom. For example, the articulation portion 608 can include a spherical shape that can allow the applicator 604 to rotate as well as pivot and thus, have more than two degrees of freedom. For instance, the body 602 can rotate about its central axis 614, which passes through the center point 612, while the applicator 604 remains in a fixed position. In addition, the applicator 604 can rotate about an axis 650, which passes through the center point 612, while the body 602 remains in a fixed position.

The articulation portion 608 and a main section 616 of the body 602 can be integral or monolithic. An indentation 618 proximate the articulation portion 608 can define a transition from the main section 616 to the articulation portion 608. The indentation 618 also can define a range of motion of the applicator 604 relative to the body 602. For example, the indentation 618 can limit the range of motion between the body 602 and the applicator 604 with respect to one degree of freedom. For instance, as shown in FIG. 6A, the applicator 604 can pivot +/−θ about the central axis 614. Angle θ can vary from about 5° to about 25°. For example, the applicator 604 can pivot about the center point 612 +/−20°.

The interior surface 610 of the body 602 can define a constriction 620. As shown in FIG. 6B, the constriction 620 can be a curved portion located proximate the articulation portion 608 formed by a decreasing internal dimension, such as an internal diameter, associated with the interior surface 610. Still consistent with embodiments disclosed herein, the constriction 620 can include the interior surface 610 forming a conical or other shape that has a larger opening proximate the coupling portion 606 than an exit proximate the articulation portion 608.

The applicator 604, as shown in FIGS. 6A and 6B, can be in the form of a nozzle. In addition, the nozzle can include a length that can be substantially longer than a diameter of an interior passage of the nozzle. As a result, during a surgical procedure, a surgeon can utilize the applicator 604 to apply bone cement to a portion of bone. Use of the apparatus 600 can participate with other accessories to pressurize the application of the bone cement such that the bone cement is forced into pores of the bone.

The body 602 and the applicator 604 can be manufactured from metals, polymers, ceramics, or any combination thereof. For example, the body 602 can be manufactured from a metal or ceramic and the applicator 604 can be manufactured from a polymer. Manufacturing the applicator 604 from a polymer or other flexible material can allow the applicator 604 to be removed from the body 602. As a result, the apparatus 600 can be a component of a kit or system that can include a plurality of applicators. As a result, a surgeon can select an appropriate applicator from a plurality of applicators for a given surgical procedure or that is sized for a particular patient.

The body 602 and the applicator 604 can be manufactured from various techniques including, but not limited to, machining, injection molding, casting, overmolding, etc.

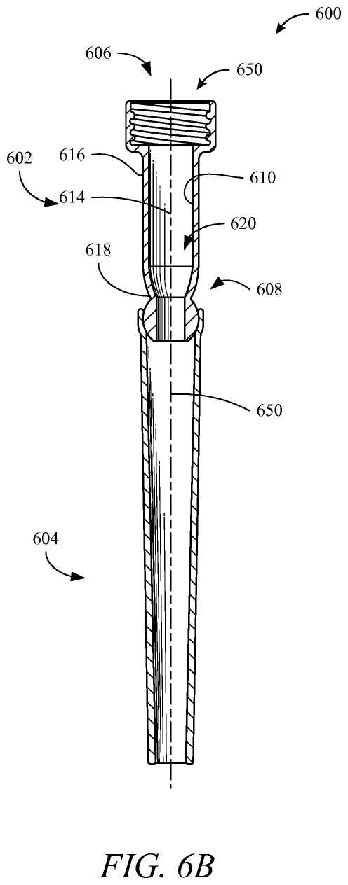

Turning now to FIGS. 7A and 7B, FIGS. 7A and 7B show an example of an apparatus 700 for applying bone cement in accordance with at least one example disclosed herein. The apparatus 700 can include a body 702 and an applicator 704. As shown in FIGS. 7A and 7B, the body 702 can include a coupling portion 706 located at a first end of the body 702. The coupling portion 706 can allow the apparatus 700 to connect to a dispenser that can contain the bone cement. As shown in FIG. 7B, the coupling portion 706 can include a groove 750. The groove 750 can receive a peg or other protrusion extending from the dispenser. Other non-limiting examples of mechanisms suitable for use as the coupling portion 706 include threads (as described with respect to FIGS. 6A and 6B), a Luer lock, male threads, press fit connections, etc.

The body 702 can also include an articulation portion 708. The articulation portion 708 can be connected to the coupling portion 706 via a through passage defined by an interior surface 710. The articulation portion 708 can include a rounded profile that can allow the applicator 704 to articulate with at least two degrees of freedom. For example, the articulation portion 708 can include a spherical shape that can allow the applicator 704 to rotate. For instance, the body 702 can rotate about its central axis 714, which passes through the center point 712, while the applicator 704 remains in a fixed position. In addition, the applicator 704 can rotate about an axis 754, which passes through the center point 712, while the body 702 remains in a fixed position.

The articulation portion 708 and a main section 716 of the body 702 can be integral or monolithic. An indentation 718 proximate the articulation portion 708 can define a transition from the main section 716 to the articulation portion 708. The indentation 718 also can define a range of motion of the applicator 704 relative to the body 702. For example, the indentation 718 can limit the range of motion between the body 702 and the applicator 704 with respect to one degree of freedom. For instance, as shown in FIG. 7A, the applicator 704 can pivot +/−θ about the central axis 714. Angle θ can vary from about 5° to about 25°. For example, the applicator 704 can pivot about the center point 712 +/−20°.

The interior surface 710 of the body 702 can define a constriction 720. As shown in FIG. 7B, the constriction 720 can be a curved portion located proximate the articulation portion 708 formed by a decreasing internal dimension, such as an internal diameter, associate with the interior surface 710. Still consistent with embodiments disclosed herein, the constriction 720 can include the interior surface 710 forming a conical or other shape that has a larger opening proximate the coupling portion 706 than an exit proximate the articulation portion 708.

The applicator 704, as shown in FIGS. 7A and 7B, can be in the form of a diffusor. As shown in FIGS. 7A and 7B, the applicator 704 can include an exit 752 that has a diameter that is larger than the constriction 720 defined by the body 702. As a result, during a surgical procedure, a surgeon can utilize the applicator 704 to apply bone cement to a portion of bone. Use of the apparatus 700 can allow the surgeon to pressurize the application of the bone cement such that the bone cement is forced into pores of the bone. For example, as the bone cement flows through the applicator 704, the velocity of the bone cement can decrease as the cross sectional area of the applicator 704 increases. The decrease in velocity of the bone cement can cause an increase pressure (static or dynamic) of the bone cement located within the applicator 704.

The body 702 and the applicator 704 can be manufactured from metals, polymers, ceramics, or any combination thereof. For example, the body 702 can be manufactured from a metal or ceramic and the applicator 704 can be manufactured from a polymer. Manufacturing the applicator 704 from a polymer or other flexible material can allow the applicator 704 to be removed from the body 702. As a result, the apparatus 700 can be a component of a kit that can include a plurality of applicators. As a result, a surgeon can select an appropriate applicator from a plurality of applicators for a given surgical procedure or that is sized for a particular patient.

The body 702 and the applicator 704 can be manufactured from various techniques including, but not limited to, machining, injection molding, casting, etc. For example, the body 702 and the applicator 704 can be injection molded. In addition, the body 702 can be machined from a cast billet using a CNC machine and the applicator 704 can be overmolded onto the articulation portion 708.

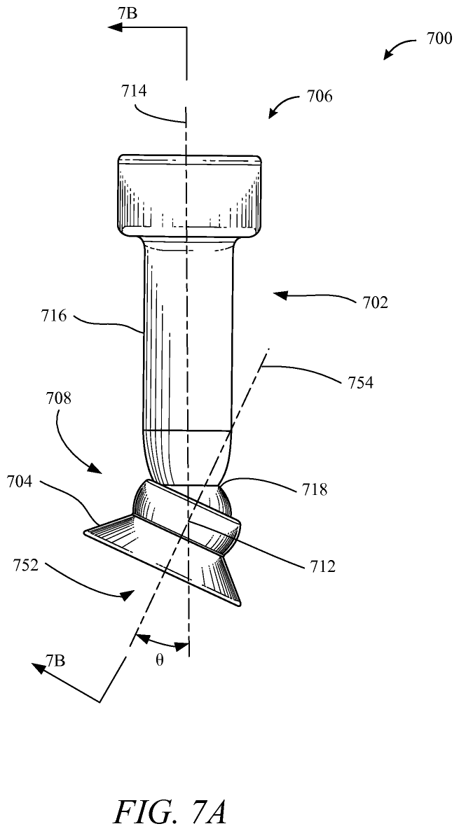

FIG. 8 shows a flowchart for a method 800 for applying bone cement in accordance with at least one example disclosed herein. The method 800 begins at stage 802 where an applicator, such as the applicator 604 or 704, of an apparatus, such as the apparatus 600 or 700, can be pivoted into a desired angle. For example, the applicator can be pivoted to a desired angle relative to a central axis, such as the central axis 614 or 714, of a body, such as the body 602 or 702. For instance, the applicator can be pivoted between 0° and 20° relative to the central axis.

From stage 802, the method 800 can proceed to stage 804 where the applicator can be placed in contact with a bone. For example, during a knee surgery, the applicator can be positioned such that an exit of the applicator is in contact with a resected surface of a bone. By pivoting the applicator relative to the body, the applicator can be place in between bones to better contact the resected surface of the bone.

From stage 804, the method 800 can proceed to stage 806 where the bone cement can be forced into pores of the resected bone. The bone cement can be forced into the pores of the bone by creating a pressure gradient within the bone cement. The pressure gradient can force the bone cement into the pores because the flow of the bone cement is restricted to a direction into the pores by the body and applicator of the apparatus.

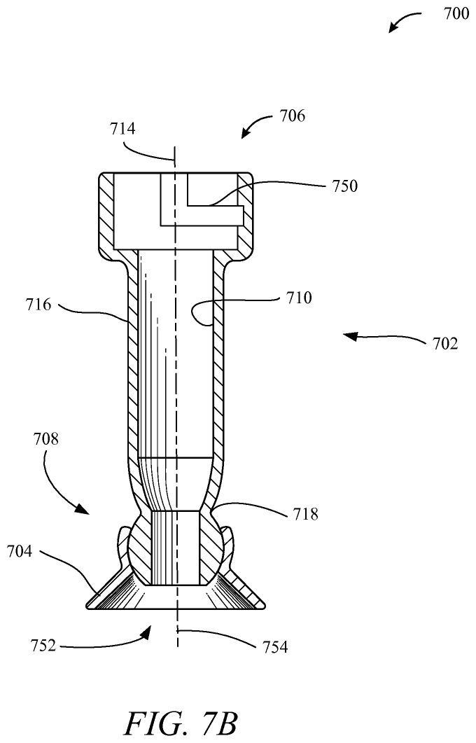

FIG. 9 shows an example of an apparatus 900 for applying bone cement in accordance with at least one example disclosed herein. The apparatus 900 can include a body 902 and an applicator 904. As shown in FIG. 9, the body 902 can include a coupling portion 906 located at a first end of the body 902. The coupling portion 906 can allow the apparatus 900 to connect to a dispenser that can contain the bone cement. For example, as disclosed herein, the coupling portion can include threads, either male or female to connect to a dispenser. Other non-limiting examples of mechanisms suitable for use as the coupling portion 906 include Luer lock, press fit connections, a grove within the body 902 that can receive a peg or other protrusion extending from the dispenser, etc.

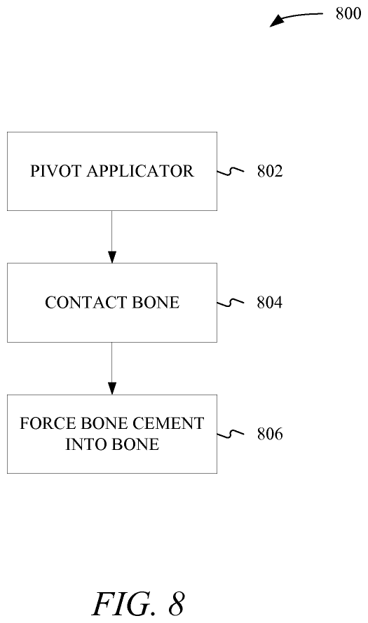

The body 902 can also include an articulation portion 910. The articulation portion 910 can be connected to the coupling portion 906 via a through passage defined by an interior surface. An applicator 912 can be connected to the articulation portion 110. As shown in FIG. 10, the applicator 912 can include a first clip 1002 and a second clip 1004. The second clip 1004 can define a groove to receive a portion of the articulation portion 910. The first clip 1002 can include a curved portion 1006. The applicator 912 can define an opening 1008. The opening 1008 can connected to the articulation portion 910 such that bone cement can pass from the body 902 through the articulation portion 910 and through the applicator 912.

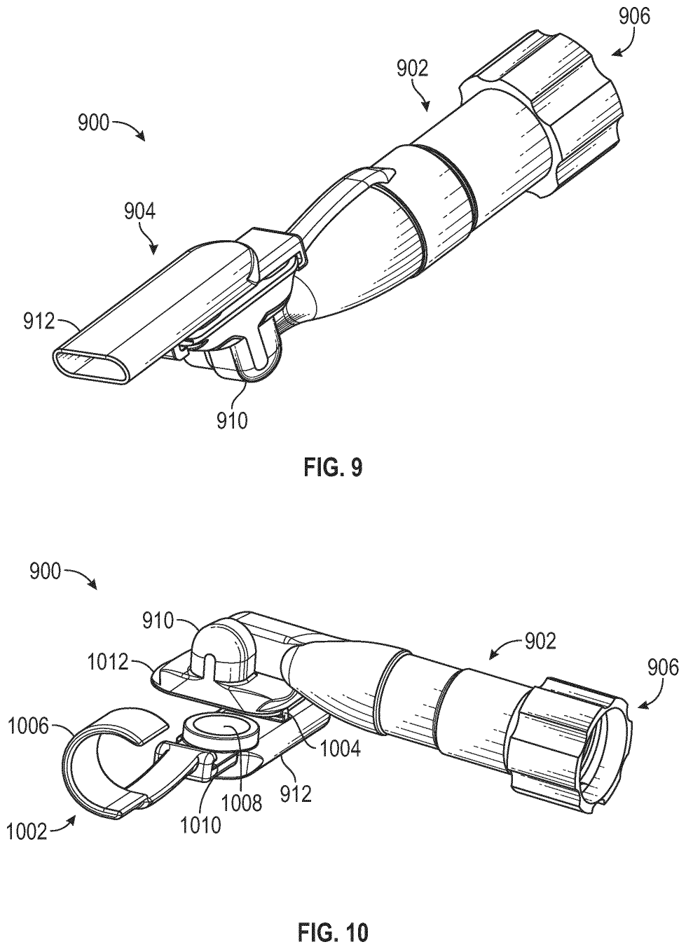

FIG. 11 shows an example assembly process for attaching the applicator 912 to the articulation portion 910. As shown in FIG. 11, the opening 1008 can be aligned with the articulation portion 910 and the applicator 912 can be rotated as indicated by arrow 1102 such that the second clip 1006 engages a grooved portion 1104. During rotation, the second clip 1004 and a third clip 1010 can engage an edge 1012 of the articulation portion. Once the second clip 1006 has engaged the grooved portion 1104, the articulation portion 910 and the applicator 912 can have a fixed position.

The body 902, articulation portion 910, and the applicator 912 can be manufactured from metals, polymers, ceramics, or any combination thereof. For example, the body 902 and the articulation portion 910 can be manufactured from a metal or ceramic and the applicator 912 can be manufactured from a polymer. The apparatus 900 can be a component of a kit or system that can include a plurality of applicators. As a result, a surgeon can select an appropriate applicator from a plurality of applicators for a given surgical procedure or that is sized for a particular patient.

The body 802, the articulation portion 910, and the applicator 912 can be manufactured from various techniques including, but not limited to, machining, injection molding, casting, etc. For example, the body 902, the articulation portion 910, and the applicator 912 can be injection molded.

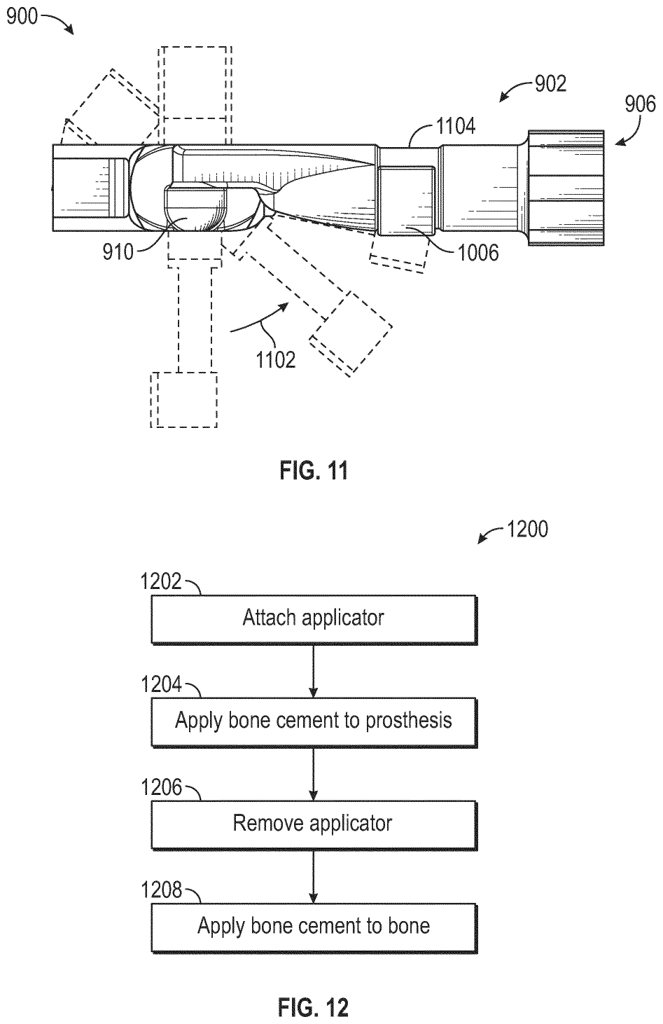

FIG. 12 shows a flowchart for a method 1200 for applying bone cement in accordance with at least one example disclosed herein. The method 1200 begins at stage 1202 where an applicator, such as the applicator 912 can be attached to an articulation portion, such as the articulation portion 910. Once the articulation portion has been attached, the method 1200 can proceed to stage 1204 where bone cement can be applied to a prosthesis. Use of the applicator can allow for a uniform application of the bone cement to the prosthesis. The uniform application of the bone cement to the prosthesis can help with setting the prosthesis. For example, the uniform layer of bone cement can allow for greater surface contact between the bone cement and the bone/prosthesis. In addition, the uniform layer of bone cement can help minimize voids within the bone cement.

From stage 1204, the method 1200 can proceed to stage 1206, where the applicator can be removed from the articulation portion. The applicator can be removed by reversing the process of attaching the applicator to the articulation portion. For example, to remove the applicator, the applicator can be rotated relative to the articulation portion so that a clip, such as the second clip 1002, can disengage the body. Once the applicator is rotated relative to the articulation portion applicator can be discarded.

From stage 1206, the method 1200 can proceed to stage 1208 where bone cement can be applied to a bone. For example, as discussed above with respect to FIGS. 4 and 5A-5E, the articulation portion can be position as needed to fit within a space created by resected bone so that bone cement can be applied. Thus, the articulation portion 910 can also be an applicator. Also, stage 1208 can be performed prior to performing stages 1202-1206. For example, bone cement can be applied to the resected bone prior to applying bone cement to the prostheses.

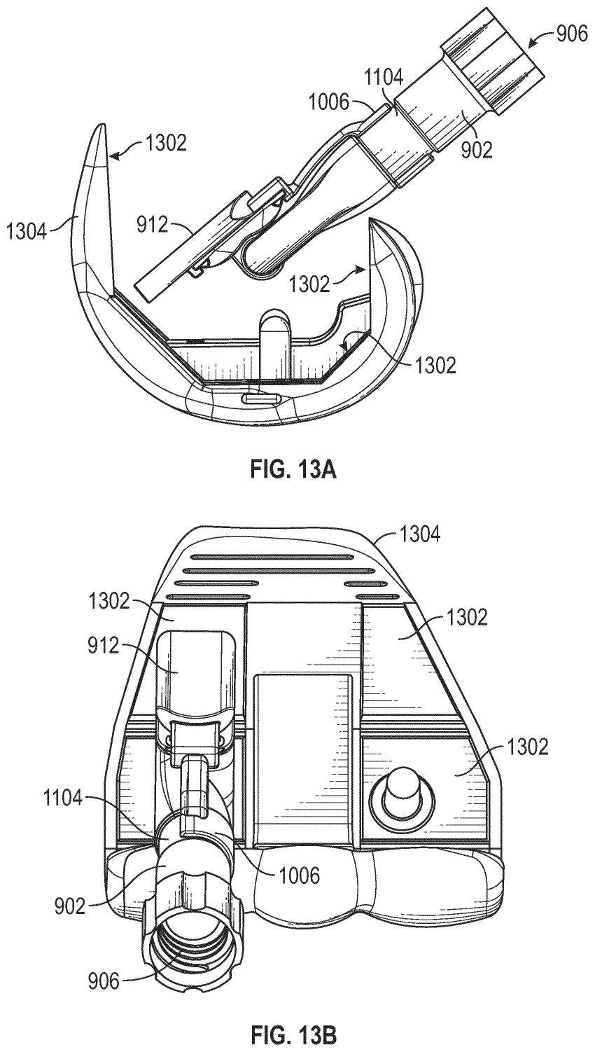

FIGS. 13A and 13B show an example process for applying bone cement in accordance with the method 1200. As shown in FIGS. 13A and 13B, the applicator 912 can be sized to correspond with one or more planar surfaces 1302 of a prosthesis 1304. The prosthesis 1304 can be a for a knee arthroplasty. For example, the prosthesis 1304 can be a prosthesis that attaches to a tibia or femur. When applying the bone cement to the prosthesis 1304, a surgeon can position the applicator 912 proximate the planar surfaces 1302 and activate the dispense to force the bone cement through the applicator 912 and onto the planar surfaces.

Once the surgeon has applied bone cement to the prosthesis, the surgeon can remove the applicator (stage 1206) and apply bone cement to prepared bone. For example, in a knee arthroplasty, the surgeon can remove the applicator from the articulation portion and apply bone cement to the resected femur and/or tibia. By removing the applicator, the surgeon can now reposition the articulation portion as described. The repositioning of the articulation portion can allow the surgeon to inject and pressurize cement in the femoral surface after the pressurization on the tibial surface, or vice versa. The size of the nozzle can allow it to fit in a gap between the tibia and femur after resection of the two bones. This can be beneficial during a knee arthroplasty where cross ligament preservation is desired.

By having one nozzle that can be used to apply bone cement to both the prosthesis and the bone, bone cement waste can be minimized because the surgeon is not having to change tools. In addition, this saves time in the operating room and helps to reduce surgery time.

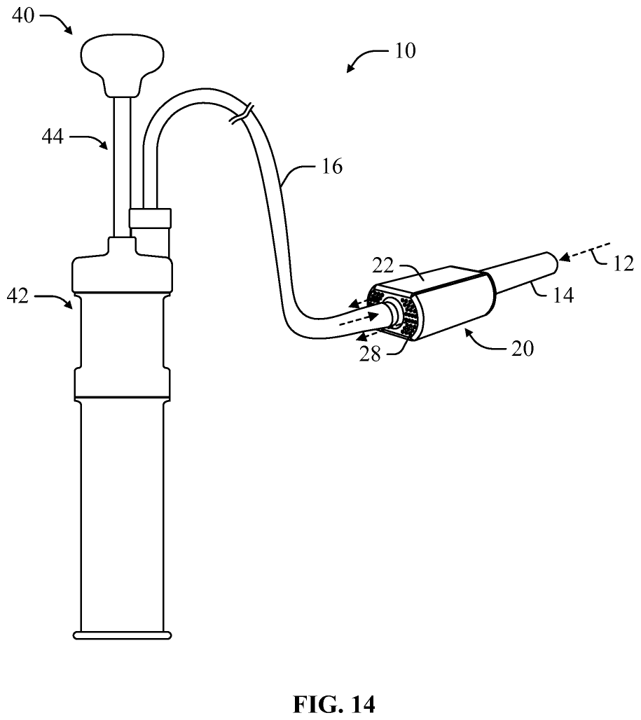

As depicted in FIG. 14, in an example, a system 10 for combining and mixing components of a bone cement can comprise a vacuum pump module 20 and a mixing device 40. The system 10 can comprise an air flow supply 12 fluidly connected to the vacuum pump module 20 via an input line 14. The vacuum pump module 20 can be fluidly connected to the mixing device 40 via a vacuum line 16. In operation, an input airflow can be provided to the vacuum pump module 20 via an input line 14 to draw a vacuum from the mixing device 40 via the vacuum line 16. The mixing device 40 can be configured to receive and mix the components of the bone cement into a paste for application to a bone or an implant.

The bone cement can comprise a liquid monomer component and a powder component such as a poly-methyl-methacrylate (PMMA) cement. The description of the bone cement is not intended to be limiting and can comprise any multi-component bone cement that can be mixed under vacuum to form a paste for application to a bone or an implant before hardening into cement.

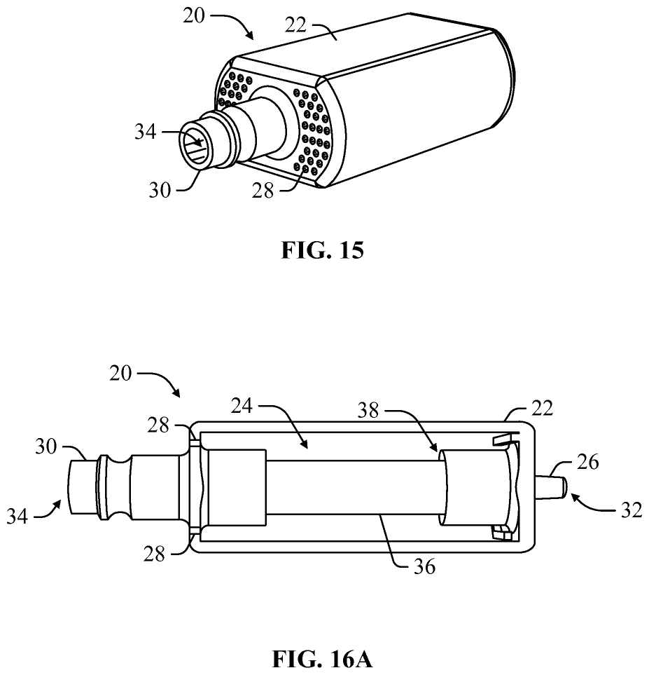

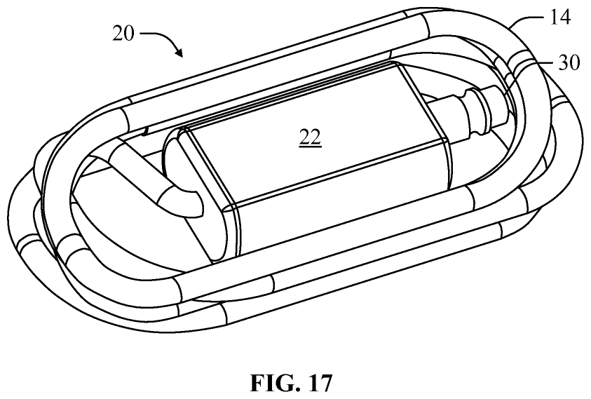

As depicted in FIGS. 14-17, in an example, the vacuum pump module 20 can include a housing 22 defining an internal chamber 24 and including an inlet connector 26, at least one outlet opening 28, and a vacuum connector 30. The inlet connector 26 can be connected to the input line 14 and define an inlet opening 32 for receiving an input airflow into the internal chamber 24 from the input line 14. The vacuum connector 30 can define a vacuum outlet 34 through the housing 22 and can be connected to the vacuum line 16. The airflow entering through the inlet opening 32 can exit the internal chamber 24 through the at least one outlet opening 28 to draw a vacuum through the vacuum outlet 34 at a predetermined pressure. In certain examples, the diameter of the inlet opening 32, the diameter and number of the vacuum outlets 34, the volume of the internal chamber 24, and combinations thereof can be varied to alter the predetermined pressure.

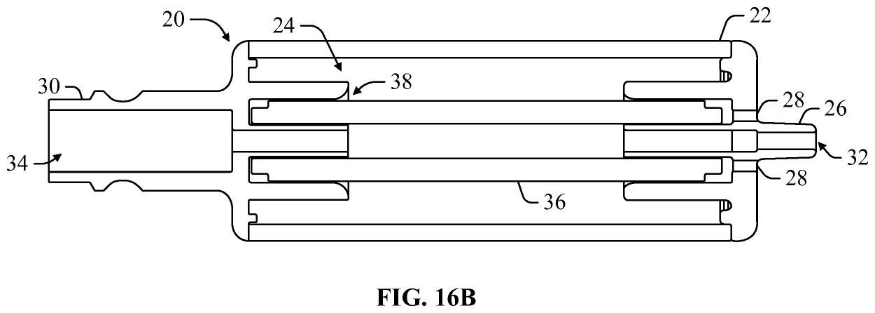

As depicted in FIGS. 16A-16B, in an example, the vacuum pump module 20 can include a Venturi element 36 defining a channel extending from the vacuum outlet 34 to proximate the inlet opening 32 of the inlet connector 26. The Venturi element 36 can define a vacuum opening 38 positioned proximate to the inlet opening 32 such that airflow entering through the inlet opening 32 draws a vacuum through the Venturi element 36 and the vacuum opening 38 through the Venturi effect. In certain examples, the vacuum opening 38 can be positioned proximate the vacuum outlet 34 as illustrated in FIG. 16B and the at least one outlet opening 28 can be positioned proximate the inlet opening 32. In certain examples, the diameter of the inlet opening 32, the diameter and length of the channel of the Venturi element 36, and combinations thereof can be varied to alter the predetermined pressure.

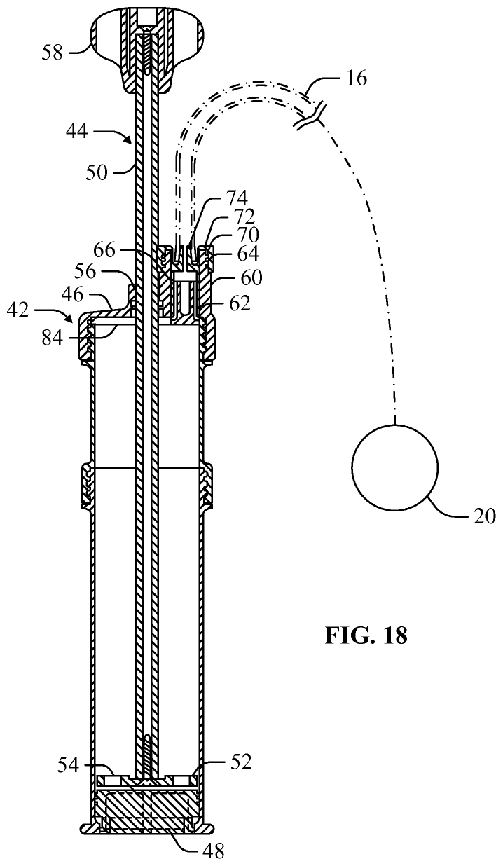

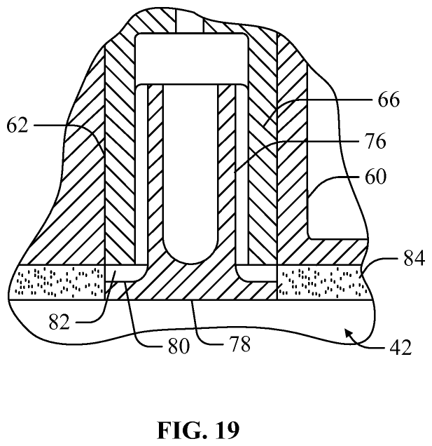

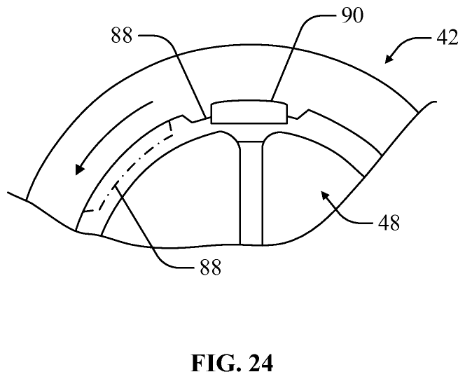

As depicted in FIGS. 18-24, the mixing device 40 can include a mixing cylinder 42 and a mixing element 44. The mixing cylinder 42 can be sealed by a cap 46 and can further include a movable member 48, such as a piston, fixed at the rear of the mixing cylinder 42 and released therefrom for movement further into the mixing cylinder 42.

The mixing element 44 can be an operating rod 50. A portion of the operation rod 50 located inside the mixing cylinder 42 can be provided with a mixing plate 52 with a plurality of through-holes 54. The operating rod 50 can protrude out of the mixing cylinder 42 through an aperture 56 in the cap 46 and the portion of the operating rod 50 situated outside the mixing cylinder 42 is provided with a handle 58.

The cap 46 can include a protruding tube section 60 configured to define the discharge opening 62 for bone cement and the other end of which can be provided with external threads 64. A plug 66 can be screwed onto the threads 64 for use when producing and collecting the bone cement, and a discharge pipe 68 can be provided for facilitating the application of the bone cement at the desired location during discharge thereof from the mixing cylinder 42. To permit screwing of the plug 66 on the threads 64 of the tube section 60, the outer end of the plug can be provided with a rearwardly directed flange 70 with internal threads 72. The outer end of the plug 66 can further comprise a connecting-pipe section 74 to which the vacuum line 16 can be connected.

The plug 66 can further comprise an inner member 76 with a bottom portion 78 configured to prevent bone cement from being forced out of the mixing cylinder 42 during the production and collection thereof. The bottom portion 78 can have or define radially directed gas passage branches 80, the inlets 82 of which lie within a filter material disk 84 of such filter material that permits penetration of gas, but not bone cement. The filter material disk 84 can be placed between an end edge of the mixing cylinder 44 and the cap 46 such that the filter material disk 84 can engage the bottom portion 78 of the inner member 76 such that only gas, but not bone cement can pass into the inlets 82 of the passage branches 80. The passage branches 80 can communicate with the connecting-pipe section 74 of the plug 66, whereby gas, using the vacuum pump module 20, can be sucked out of the mixing cylinder 42 through the filter material disk 84.

When bone cement is ready-mixed and collected in the mixing cylinder 42, the plug 66 can be unscrewed, whereby the discharge opening 62 in the protruding tube section 60 is exposed. The discharge pipe 68 can then be screwed onto the threads 64 of the protruding tube section 60, which permitted by the internal threads 86 on the discharge pipe 68.

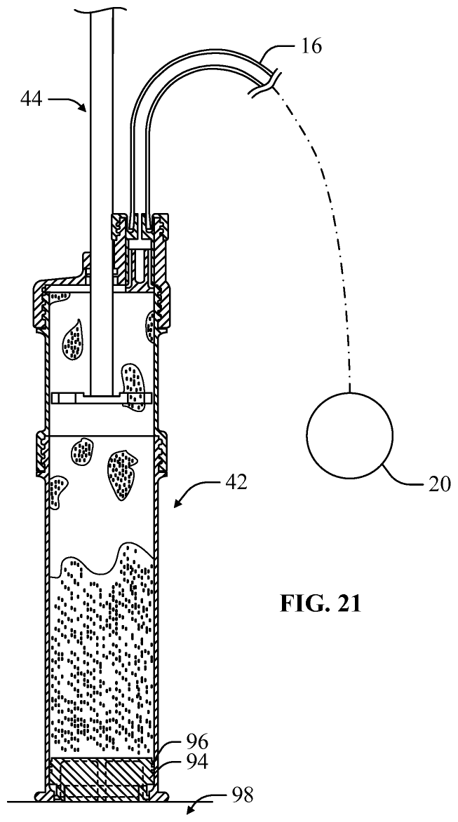

The movable piston 48 can be fixed at the rear of the mixing cylinder 42 such that the movable piston 48 does not move when mixing of bone cement occurs in the mixing cylinder 42. The piston 48 can be releasable and configured such that the piston 48 can be brought to move farther into the mixing cylinder 42 when there is a vacuum therein for collecting the ready-mixed bone cement at the front of the mixing cylinder 42 closest to the discharge opening 62. To enable this fixing and release of the piston 48, the mixing cylinder 42 can have at the rear at least one oppositely directed gripping portion 88, which e.g. can have the shape of a flange which extends only along a portion of the inner periphery of the mixing cylinder 42. The piston 48 on the other hand, can have at least one corresponding gripping portion 90 shaped as an outwardly directed lip which can protrude from the gripping portion 88 of the mixing cylinder 42. The lip can define a groove with the outer side of the piston into which the gripping portion 88 of the mixing cylinder 42 can extend, and the lip can also be shaped such that the lip extends only along a limited portion of the outer periphery of the piston 48. By this gripping structure, the piston 48 can be kept fixed to the rear portions of the mixing cylinder 42 and the piston 48 can be released by rotation relative to the mixing device or vice versa until the gripping portions 88, 90 are disengaged (see position of the gripping portion 88 of the mixing cylinder 42 indicated with dashed and dotted lines in FIG. 21).

To enable release of the piston 48 by simple means and in a simple manner, the piston 48 can have gripping surfaces 92, e.g. one or more recesses on the outer side of the piston 48, which can be brought in engagement with corresponding gripping surfaces 94, e.g. one or more upwardly directed pins, on a separate member 96, located on a base. This separate member 96 can, e.g. be a trough, which can be placed on the operating table. By positioning the mixing cylinder 42 on the separate member 96 such that the gripping surfaces 92 of the piston 48 and the gripping surfaces 94 on the separate member 96 cooperate with each other, the piston 48 can be released by rotating the mixing cylinder 42 relative to the separate member 96, since the gripping surfaces 94 of the separate member 96 prevent the piston 48 from rotating with the mixing cylinder 42.

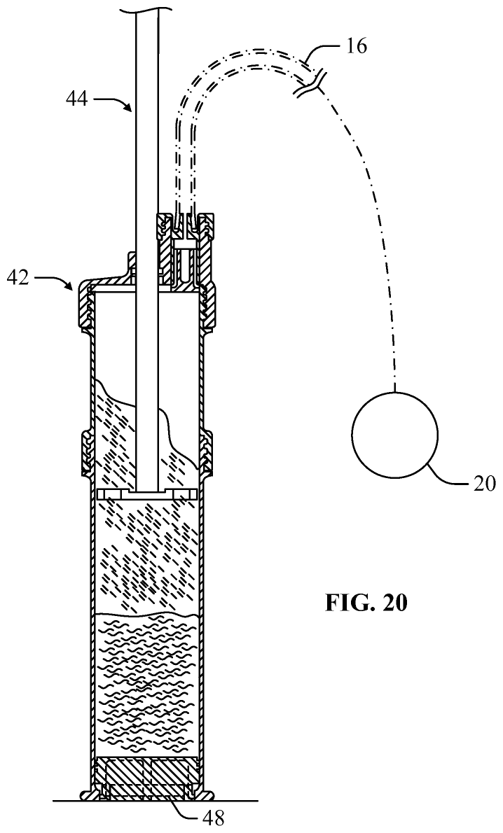

For producing and collecting bone cement of the present type, an appropriate amount and type of components can be filled into the mixing cylinder 42 (see FIG. 20). The mixing cylinder 42 can then be connected to the vacuum pump module 20 until a predetermined vacuum is generated in the cylinder. Mixing of the components can occur by moving the operating rod 50 up and down in the mixing cylinder 42, whereby the bone cement passes through the holes 10 in the mixing plate 52. This procedure can be continued until the components are mixed sufficiently for producing bone cement having predetermined properties.

When the mixing step is concluded, the bone cement is not gathered in the front or fore portions of the mixing cylinder 42. Instead, the bone cement can be spread in different portions of the mixing cylinder 42 and the bone cement can adhere in clumps to the inner surfaces of the mixing cylinder 42, to the mixing plate 52 and eventually also to the operating rod 50 (see FIG. 21) particularly if the viscosity of the bone cement is high. Collection of the bone cement can occur by generating a vacuum in the pores formed in the bone cement during collection or gathering. Hereby, the volume of porosity in the bone cement can be reduced when subjected to atmospheric pressure after gathering, e.g. when the vacuum pump module 20 is disconnected from the mixing cylinder 42 such that air may flow into the cylinder. Since there is a vacuum in the pores formed during collection or gathering, the volume of porosity in the bone cement is reduced e.g. by the disappearance of smaller pores and a reduction in the volume of other pores.

The bone cement can be collected in a vacuum which is maintained after the mixture of the bone cement is ready. Hereby, it is possible during collection or gathering either to maintain the same vacuum as during mixing or to increase or decrease the vacuum present during mixing.

The bone cement can be collected or gathered in at least 40% vacuum and preferably in 80-95% vacuum.

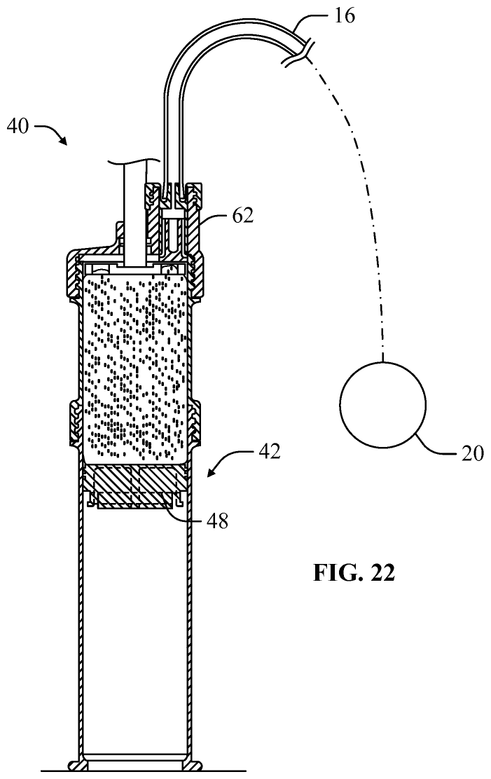

The collection of bone cement can be facilitated by the piston 48, which after release (see FIG. 22), is drawn into the mixing cylinder 42 while there is a vacuum therein, whereby bone cement can be collected at the front in the mixing cylinder.

Gas, but not bone cement, can pass through the filter material disk 84 to the vacuum pump module 20 during collection.

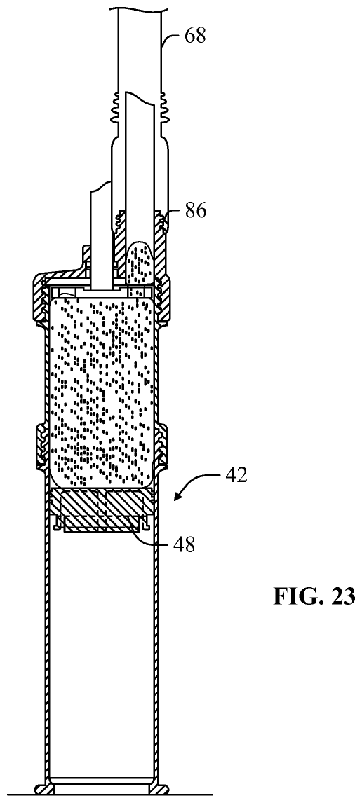

After collection of the bone cement, the vacuum 16 can be disconnected, the operating rod 50 (which thereby is situated in its upper-end position) can be broken and the plug 66 removed, whereafter the discharge pipe 68 is connected to the mixing cylinder 42 (see FIG. 23). The mixing device 40 can now be used for application of the finished and collected bone cement. The discharge of the bone cement from the mixing cylinder 42 through the discharge opening 62 and discharge pipe 68 can be performed by placing the mixing cylinder 42 in a so-called outfeed or discharge pistol (not shown), which is designed such that it can affect the piston 48 in forward direction, whereby the piston 48 is pressing or forcing the bone cement out of the mixing cylinder 42.

It will be readily understood to those skilled in the art that various other changes in the details, material, and arrangements of the parts and method stages which have been described and illustrated in order to explain the nature of the inventive subject matter may be made without departing from the principles and scope of the inventive subject matter as expressed in the subjoined claims.

1. An apparatus for applying bone cement, the apparatus comprising:
a body including a coupling portion located at a first end of the body and an articulation portion located at a second end of the body, a central axis of the articulation portion arranged at an angle relative to a central axis of the body; and
an applicator configured to connect to the articulating portion and pivot about the central axis of the articulation portion.

2. The apparatus of claim 1, wherein the applicator is configured to pivot at least 180 degrees about the central axis of the articulation portion.

3. The apparatus of claim 1, wherein the central axis of the articulation portion is perpendicular to the central axis of the body.

4. The apparatus of claim 1, wherein an interior surface of the body defines a constriction proximate the articulation portion.

5. The apparatus of claim 1, wherein the applicator includes a diffusor.

6. The apparatus of claim 1, wherein the applicator includes a central axis arranged at an angle relative to the central axis of the articulation portion.

7. The apparatus of claim 6, wherein the angle of the central axis of the articulation portion relative to the central axis of the body is about 90 degrees.

8. The apparatus of claim 1, wherein the applicator includes an interior surface at least partially contoured to match a contour of a resected bone.

9. The apparatus of claim 1, wherein the applicator is selected from a plurality of applicators, each of the plurality of applicators having at least one of a different size or a different shape.

10. A system for producing reduced porosity bone cement, comprising:
a vacuum pump module comprising:
a housing defining an internal chamber and at least one outlet opening,
an inlet connector defining an inlet opening in communication with the internal chamber, and
a vacuum connector defining a vacuum outlet in communication with the internal chamber,
wherein the vacuum pump module is configured to define an airflow path through the inlet opening into the internal chamber, and through the at least one outlet opening to draw air through the vacuum outlet; and
a mixing device including a mixing container defining a mixing space;
wherein the mixing container is fluidly connected to the vacuum connector such that drawing air through the vacuum outlet creates a vacuum within the mixing space at a predetermined pressure.

11. The system of claim 10, wherein the vacuum pump module can further comprise:
a venturi element defining a channel extending through the internal chamber from the vacuum outlet to a vacuum opening;
wherein the vacuum opening is proximate to the inlet opening such that the airflow entering through the inlet opening and passing the vacuum opening draws air through the channel and from the vacuum outlet.

12. The system of claim 10, wherein the mixing space has wall surfaces to which clumps of bone cement adhere during mixing of components of the bone cement, and wherein the mixing device further comprises:
means for mixing the components in the mixing space;
a front member provided at a front end of a mixing cylinder, the front member having a discharge opening for discharging bone cement from the mixing space; and
a movable piston fixedly mounted at a rear end of the mixing cylinder, the movable piston being releasable from the rear end of the mixing cylinder and movable in the mixing space toward the discharge opening for collecting the clumps of bone cement adjacent the discharge opening, and for discharging bone cement through the discharge opening;
wherein, when the movable piston is released from the rear end of the mixing cylinder and moved in the mixing space, the vacuum pump module generates a vacuum in pores formed in the bone cement during the collecting of the clumps of bone cement, whereby the size of the pores is reduced when the bone cement, after collecting, is exposed to atmospheric pressure.

13. The system of claim 12, wherein the movable piston comprises at least one gripping portion and the mixing cylinder comprises at least one corresponding gripping portion, the gripping portions of the movable piston and the mixing cylinder are engageable with each other for fixing the movable piston to the mixing cylinder, and the gripping portions are disengagable from each other by at least one of: rotating the movable piston relative to the mixing cylinder and rotating the mixing cylinder relative to the movable piston.

14. The system of claim 13, wherein the gripping portions are disengageable from each other by rotating the mixing cylinder relative to the movable piston after the movable piston is placed into engagement with a separate member, wherein the separate member prevents the movable piston from rotating when the mixing cylinder is rotated relative to the separate member.

15. The system of claim 12, wherein a discharge pipe for discharging bone cement from the mixing space is provided on a tube section formed on the front member, wherein the discharge pipe is provided with internal threads for engagement with external threads provided on the tube section.

16. A method for applying bone cement, the method comprising:
pivoting an applicator into a desired angle relative to central axis of an articulation portion, the articulation portion connected to a body at a 90 degrees angle, the articulation portion connected to the applicator;
contacting a surface of a first bone with the applicator; and
forcing the bone cement into pores of the first bone via a pressure generated within the body due to the applicator being in contact with the surface of the first bone.

17. The method of claim 16, wherein pivoting the applicator to the desired angle includes pivoting the applicator to an angle between +/−90 degrees relative to a central axis of the body.

18. The method of claim 16, further comprising selecting the applicator from a plurality of applicators.

19. The method of claim 16, further comprising placing the applicator in between the first bone and a second bone, the first bone and the second bone adjacent to one another.

20. The method of claim 19, wherein a first bone is a femur and a second bone is a tibia.
